(19) United States
(12) Patent Application Publication
(10) Pub. No.: US 2002/0068870 A1
(43) Pub. Date: Jun. 6, 2002

(54) Hand held mechanical compression device for inducing tissue strain

(76) Inventors: Sheikh Kaisar Alam, Somerset, NJ (US); Ernest J. Feleppa, Rye, NY (US); Marvin King, New York, NY (US); Frederic L. Lizzi, Tenafly, NJ (US)

Correspondence Address: BAKER & BOTTS, 30 ROCKEFELLER PLAZA, NEW YORK, NY, 10112

(21) Appl. No.: 09/910,621

(22) Filed: Jul. 20, 2001

Related U.S. Application Data
(60) Provisional application No. 60/219,517, filed on Jul. 20, 2000.

(51) Int. Cl. A61B 8/00

(52) U.S. Cl. 600/446

(57) ABSTRACT

An apparatus for ultrasonic elastography includes a housing, a moveable surface, and a transducer in ultrasonic communication with the moveable surface. A driver is arranged to mechanically move the moveable surface with respect to the housing. The housing can also be formed such that the moveable surface is a moveable exterior surface of the housing, such as a compliant membrane, which is moved in response to the driver to induce tissue compression. The housing may be arranged to be held in an operator's hand. Alternatively, the housing may be arranged for an insertion into an opening in a patient's body. The driver may move the transducer or moveable exterior surface in a cyclical manner at a low frequency.

### SPECIFICATION

This application claims the benefit of U.S. Provisional application, Ser. No. 60/219,517, entitled Imaging of Radioactive Seeds for Radiation Therapy of the Prostate, filed on Jul. 20, 2000.

### BACKGROUND OF INVENTION

This invention relates to ultrasonic elasticity imaging devices, and more particularly to a handheld mechanical compression device for inducing controlled tissue strain.

Ultrasound based elasticity imaging methods produce images that convey information regarding tissue elastic properties, as opposed to information regarding tissue acoustic scattering properties conveyed by conventional b-mode ultrasonograms. One of the ultrasonic elasticity imaging methods is elastography. Elastography produces high resolution elastograms (elastographic images) that quantitatively depict local tissue deformation under quasi-static external compression. Tissue deformation can be quantitatively described by strain which is defined as the change in length divided by the length of a section of tissue in a given direction. Elastograms may be generated as follows:

(1) A frame of RF echo signals from tissue is digitized before compression;

(2) A small quasi-static compression is applied on the tissue along the axis of the transducer by a computer controlled fixture;

(3) A second RF frame is digitized after compression; and

(4) The acquired pre- and post-compression RF echoes are analyzed to compute the induced tissue strain.

FIGS. 1A through 1C illustrate the principals of elastography using an exemplary 3-layer object having a soft bottom layer, a rigid middle layer and a very soft top layer. As illustrated in FIGS. 1B and 1C, when this object is subjected to external compression, the rigid middle layer undergoes virtually no strain whereas the top softest layer experiences the largest strain.

In typical elastography implementations the required compression is applied using a large computer controlled mechanical fixture. Internal organs, such as the prostate, are not accessible to such devices. Free-hand application of compression induces large and irregular tissue motion leading to significant, frequently unrecoverable, errors in conventional elastography. Accordingly, there is a need for a handheld device for applying compression on tissue which allows acceptable strain estimation with conventional methods. It is an object of the present invention to provide new and improved mechanical devices for elastography.

### SUMMARY OF THE INVENTION

In accordance with the invention there is provided a hand held apparatus for ultrasonic elastography which includes a housing, a moveable surface, a transducer mounted to the housing which is in ultrasonic communication with the moveable surface and a driver which is arranged to mechanically move the moveable surface with respect to the housing.

In a preferred arrangement, the housing is arranged to be held in an operator's hand. The driver is preferably arranged to move the transducer in a direction which is substantially orthogonal to the emitting surface. Preferably, the driver is arranged to move the transducer cyclically at a low frequency, such as less than 20 hertz. The cyclical motion may be in a sinusoidal manner or alternately the driver may be arranged to move the transducer with substantially constant displacement velocity. The driver can also be arranged to induce a single compression at various compressive forces during which multiple RF data frames can be acquired.

In accordance with the invention there is provided an apparatus for ultrasonic elastography which includes a housing having at least one exterior moveable surface. A transducer is mounted within the housing for transmitting and receiving ultrasonic signals though the moveable exterior surface and a driver is arranged to move the exterior surface.

In a preferred arrangement the exterior moveable surface is a compliant membrane. In one arrangement, the transducer is an elongated structure for insertion into an opening in a patient's body and a membrane substantially surrounds at least one end of the elongated structure. The membrane may be a membrane covering a planar opening of the housing. In one embodiment, the membrane may be more compliant in an axial direction with respect to the transducer than in a radial direction with respect to the transducer. In one arrangement, fluid is provided between the elongated structure and the membrane and the driver is arranged to vary the amount of the fluid. In another arrangement fluid may be provided in an enclosure and the driver may vary the amount of fluid in the enclosure to move the moveable exterior surface.

In accordance with the invention there is provided a method for obtaining tissue characteristics with an assembly that includes a transducer assembly having a moveable surface. The surface is applied against a body portion corresponding to tissue to be evaluated. The surface is moved toward and away from the tissue to compress the tissue. Ultrasonic signals are transmitted and received from the tissue in synchronism with moving of the surface and the signals are evaluated to derive tissue characteristics.

In one preferred arrangement of the method the surface of the transducer assembly is manually placed against the skin of the patient. In another arrangement the surface of the transducer assembly is inserted into an opening in a patient's body.

For a better understanding of the present invention, together with other and further objects, reference is made to the following description, taken in conjunction with the accompanying drawing, and its scope will be pointed out in the appended claims.

### BRIEF DESCRIPTION OF THE DRAWINGS

FIG. 1A is a cross sectional view of an exemplary 3-layer object having a soft bottom layer, a rigid middle layer and a very soft top layer prior to application of a compressive force.

FIG. 1B is a cross sectional view of an exemplary 3-layer object having a soft bottom layer, a rigid middle layer and a very soft top layer following application of a compressive force.

FIG. 1C is a graph illustrating a strain profile for the 3-layer object of FIGS. 1A and 1B.

FIG. 2 is a cross sectional view of a first embodiment of a hand held apparatus for performing ultrasonic elastography.

FIG. 3 is a schematic diagram illustrating an alternate arrangement for an apparatus for performing elastography according to the invention.

FIGS. 4 and 5 are cross sectional views illustrating an apparatus for performing elastography according to the invention which is particularly adapted for examining tissue which is adjoining a cavity in a patient's body.

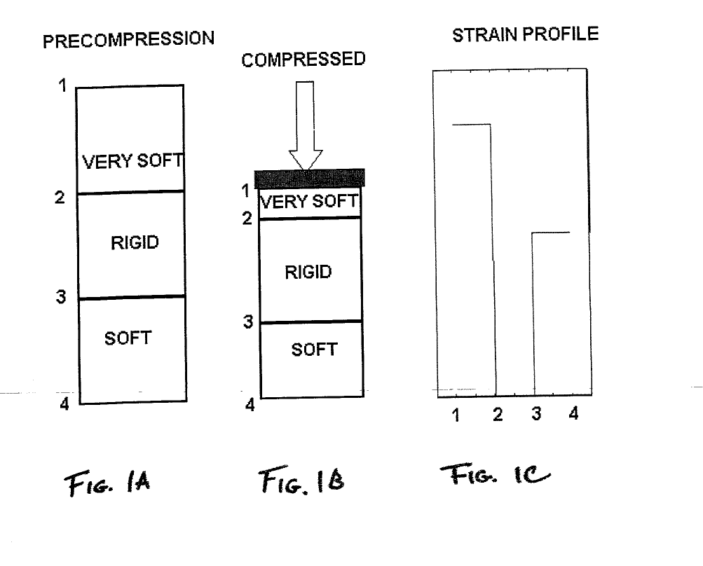

### DESCRIPTION OF THE PREFERRED EMBODIMENTS

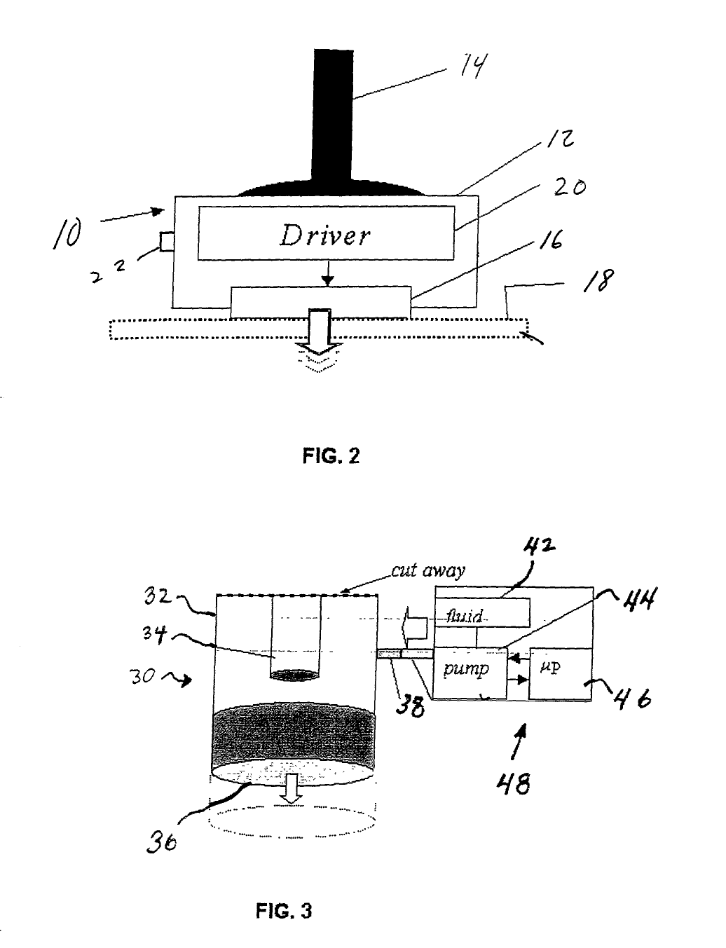

The apparatus 10 shown in FIG. 2 includes a housing 12 which is provided with an optional handle 14 to be held by the person performing the examination. Housing 12 encloses at least a portion of a transducer 16 for applying ultrasonic signals to a patient. A flat plate 18 may also be provided to provide a larger surface area of compression thereby providing a more uniform stress distribution. The flat plate 18 may be a solid plate formed from an ultrasonically transmissive material and interposed between the surface of the transducer and the patient. Alternatively, the plate 18 may have an opening through which the transducer 16 extends such that the surface of the transducer 16 is substantially coplanar with the surface of the plate 18 which will be placed against the patient.

A driver 20 is provided within housing 12 for mechanically moving transducer 16 and plate 18 (if provided) to induce compression in the tissue being examined. An examination may be performed by holding the plate 18 against a patient's skin and activating a button 22 which causes driver 20 to move the transducer 16 and plate 18 to provide compressive force to the tissue being examined. Preferably, the direction of compression is substantially orthogonal to the surface of the transducer 16 and plate 18. Transducer 16 is arranged to provide transmission and reception of ultrasonic signals through plate 18 into the tissue of a patient so that the tissue may be examined during the compressive movement of transducer 16 and plate 18. In one arrangement driver 20 may be for example, a mechanical motor or a stepper motor which drives the transducer 16 and plate 18 in a sinusoidal or linear cyclical motion, such as a sine wave or a triangular wave. The driver can also be arranged to provide a single compression of varying force. In this case, multiple sets of ultrasound data can be acquired at varying degrees of compression.

While driver 20 is moving transducer 16, ultrasonic signals are recorded prior to and during compression of the tissue to derive elastographic information concerning the tissue being examined. The apparatus 10 of FIG. 2 can be arranged to be held by the optional handle 14 during examination of tissue. Alternatively, the housing 12 can be sized and shaped to fit in the hand of a user.

FIG. 3 shows an alternate arrangement for the apparatus according to the invention. The FIG. 3 apparatus is cut away such that the optional handle 14 is not visible. The FIG. 3 apparatus 30 includes a housing 32 within which is mounted an ultrasonic transducer 34. Housing 32 is generally hollow and contains fluid. The lower end of housing 32 is closed by a membrane 36, which may be stretched across a planar opening. A driver unit 40 is arranged to change the amount of fluid in housing 32. In one embodiment, the driver unit 40 can take the form of a syringe which is manually compressed to alter the fluid volume in housing 32. Alternatively, the driver unit 40 can include a fluid reservoir 42, a pump 44 and may include a microprocessor 46 for controlling the operation of pump 44. Pump 44 is connected to the interior of housing 32 through a conduit 38. In the apparatus of FIG. 3, the membrane 36 is applied against the tissue of a patient and the fluid is provided to housing 32 by pump 44 (or a syringe) to move membrane 36 away from transducer 34 to compress the tissue while transducer 34 sends and receives ultrasonic signals.

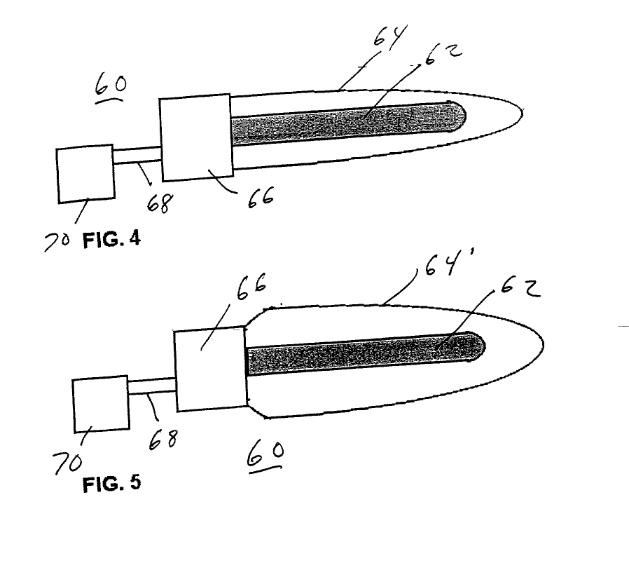

FIGS. 4 and 5 illustrate an apparatus according to the invention which is particularly adapted for examining tissue which is adjoining a cavity in a patient's body. For example, the apparatus 60 and FIGS. 4 and 5 may be used to provide ultrasonic elastographic examination of the prostate gland by inserting the apparatus into the rectum and causing the apparatus to compress the tissue as will be described. Apparatus 60 includes a base 66 on which there is mounted a cylindrical transducer 62 of the type currently used for transrectal ultrasound, such as in prostate examination. A membrane 64, such as a condom, surrounds transducer 62 and is secured at the base of transducer 62 so as to establish a fluid containing vessel. Conventional transrectal ultrasound transducers are commonly provided with a fluid inlet in the base and a fluid outlet at the opposite end. This allows fluid to be provided to the space between membrane 64 and transducer 62, such as by a syringe. In conventional transrectal ultrasound, the introduction of the fluid is used to establish an ultrasonic interface between the rectal cavity and the transducer. Fluid can also be introduced by a pump 70 which is in fluid communication with the interior of membrane 64 by a conduit 68.

As shown in FIG. 5 the addition of fluid to the space between the transducer 62 and membrane 64 causes the expansion of the membrane. The expansion of the membrane 64 as shown causes compression of the tissue of interest. The membrane can be formed such that expansion is primarily directed in a direction orthogonal to the axis of transducer 62, such as by forming the end with substantially less compliance than the sidewalls. This can be achieved by forming the end with a material with different elastic properties or with a different material thickness to limit expansion along the axis of the transducer.

In the embodiments of the invention described herein, the driver can be selected and arranged to induce low frequency cyclical motion or vibration. The frequency of this cyclical motion, and at which compression is applied, is ordinarily below 10 Hz to provide a quasi-static measurement condition. For example, the handheld mechanical compression device, such as exemplified in FIG. 2, can be provided with a driver 20 for displacing the transducer 16 and/or plate 18 in a low-frequency-cycled manner.

At the beginning of a tissue strain estimation process, the handheld mechanical compression device 10 is placed against the tissue to be analyzed. When the handheld mechanical compression device is activated, the transducer 16 initially collects a pre-compression frame of RF signal data in response to a signal from a controller (not shown). The handheld mechanical compression device 10 may be activated by a switch 22 or by other means known in the art. The driver 20 then automatically displaces the transducer 16 and/or plate 18 in a low frequency cyclical manner, such as with a sinusoidal motion, thus inducing a cyclical tissue strain. The transducer 16 then collects post-compression RF echo signal data at predetermined points of the compression cycle. This can be achieved by synchronizing the operation of the transducer 16 and the driver 20 using a controller (not shown), such as a microprocessor.

At times near the zero-crossings of the sinusoidal cyclical compression, displacement from applied low frequency vibration is substantially linear and the applied displacement within a fixed time interval is at a maximum. If a data frame sequence is acquired in this region, strain can readily be computed using elastographic signal processing methods. Data acquisition can be synchronized to the vibration driver to ensure that data is acquired in the right time interval. While not preferred, data can be acquired at less linear portions of the sinusoidal compression cycle so long as the data are acquired at known points of the sinusoidal motion.

Alternatively, in place of a sinusoidal displacement from the driver 20 to induce tissue compression, a low frequency triangular waveform or reverse saw-tooth waveform, can also be used to induce tissue strain. An advantage of such waveforms is that the rate of change is substantially constant within a linear portion of the curve. However, such waveforms require the use of a driver 20 which can accommodate such triangular motion (e.g., stepper-motor based).

In one embodiment using low frequency vibration to induce tissue compression, the driver 20 can take the form of a conventional audio speaker driven by an appropriately shaped driving signal, such as from a function generator. Alternatively, precise vibration devices (e.g., mini-shaker type 4810 by Bruel & Kjaer, Denmark) or stepper-motor controlled systems can also be used.

According to the method of the present invention, an apparatus as described herein is applied against the tissue of the patient and held in place by hand or otherwise. The apparatus is used to apply strain to the tissue and ultrasonic data is recorded prior to and following the application of strain such that the tissue may be characterized in terms of its elastic properties. In addition, the driver can be arranged to Japply a variable degree of force during a single compression and multiple sets of ultrasonic data can be acquired during such a compression.

While there have been described what are believed to be the preferred embodiments of the present invention, those skilled in the art will recognize that other and further changes may be made thereto without departing from the spirit of the invention and it is intended to claim all such changes and modifications as fall within the scope of the invention.

We claim:

1. A hand-held apparatus for ultrasonic elastography, comprising:
a housing;
a moveable surface;
a transducer mounted in said housing and being in ultrasonic communication with said moveable surface; and
a driver in said housing and arranged to mechanically move said moveable surface with respect to said housing.

2. A hand-held apparatus as specified in claim 1 wherein said housing is sized and shaped to be held in an operator's hand.

3. A hand-held apparatus as specified in claim 1 further comprising a handle attached to said housing.

4. A hand-held apparatus as specified in claim 1 wherein said driver is arranged to move said moveable surface in a direction which is substantially orthogonal to said moveable surface.

5. A hand-held apparatus as specified in claim 1 wherein said driver is arranged to move said moveable surface cyclically at a low frequency.

6. A hand-held apparatus as specified in claim 4 wherein said low frequency is less than 20 Hz.

7. A hand-held apparatus as specified in claim 4 wherein said driver is arranged to move said moveable surface in a sinusoidal manner.

8. A hand-held apparatus as specified in claim 4 wherein said driver is arranged to move said moveable surface with substantially constant displacement velocity.

9. Apparatus for ultrasonic elastography, comprising:
a housing having at least one moveable exterior surface;
a transducer mounted within said housing for transmitting and receiving ultrasonic signals through said moveable exterior surface; and
a driver arranged to move said exterior surface.

10. Apparatus as specified in claim 9 wherein said moveable exterior surface comprises a compliant membrane.

11. Apparatus as specified in claim 10 wherein said housing defines a substantially planar opening and wherein said compliant membrane covers said planar opening of said housing.

12. Apparatus as specified in claim 10 wherein said transducer comprises an elongated structure for insertion into an opening in a patient's body, and wherein said membrane surrounds at least one end of said elongated structure.

13. Apparatus as specified in claim 12 wherein said driver is a fluid pump in fluid communication with said housing and wherein said driver varies the amount of said fluid to move said exterior surface.

14. Apparatus as specified in claim 12 wherein said membrane is more compliant in an axial direction with respect to said elongated structure than in a radial direction with respect to said elongate structure.

15. Apparatus as specified in claim 9 wherein said enclosure contains fluid, and wherein said driver varies the amount of fluid in said enclosure to move said moveable exterior surface.

16. A method for obtaining tissue characteristics comprising:
providing a hand held transducer assembly having a moveable surface;
applying said surface against a body portion corresponding to tissue to be evaluated;
moving said surface toward and away from said tissue to compress said tissue;
transmitting and receiving ultrasonic signals from said tissue in synchronism with said moving of said surface; and
evaluating said signals to derive tissue characteristics.

17. A method according to claim 16 wherein said applying comprises manually placing said transducer assembly surface against the skin of a patient.

18. A method according to claim 16 wherein said applying comprises inserting said transducer assembly surface into an opening in a patient's body.

19. A method according to claim 16 wherein said moving said surface comprises moving said surface in a low frequency cyclical manner.

20. A system for ultrasonic elastography, comprising:
a controller;
a housing;
a transducer mounted in said housing and having an ultrasonic emitting surface, said transducer transmitting ultrasound signals and receiving echo signals in response to said controller; and
a driver in said housing and arranged to mechanically move said emitting surface with respect to said housing in a low frequency cyclical manner in response to a signal from said controller.

21. The system for ultrasonic elastography of claim 20, wherein said controller receives echo signals from said transducer at predetermined intervals with respect of the low frequency movement.

22. The system for ultrasonic elastography of claim 21, wherein the low frequency movement is substantially sinusoidal and wherein said predetermined intervals substantially correspond with zero crossings of the sinusoidal movement.

23. The apparatus of claim 21 wherein the low frequency movement is a substantially triangular cyclical movement.

24. The system for ultrasonic elastography of claim 20, wherein said driver comprises a speaker operatively coupled to a driving waveform signal.

25. The system for ultrasonic elastography of claim 20, wherein said driver comprises a precision shaker.

26. The system for ultrasonic elastography of claim 20, wherein said driver comprises a stepper motor.

27. A system for ultrasonic elastography comprising:
a fluid containing housing having at least one compliant surface;
an ultrasound transducer, said ultrasound transducer being disposed within said fluid containing housing and in ultrasonic communication with said compliant surface;
a fluid pump, said fluid pump being in fluid communication with said fluid containing housing; and
a controller, said controller being operatively coupled to said fluid pump and said ultrasonic transducer, said controller operating said transducer to acquire a pre-compression transducer signal, operating said pump to increase a volume of fluid in said fluid containing housing to deform said compliant surface, and operating said transducer to acquire a post-compression transducer signal.

28. The system for ultrasonic elastography according to claim 27, wherein said housing is a hand-held housing and said compliant surface is a substantially planar surface.

29. The system for ultrasonic elastography according to claim 27 wherein said transducer is an elongate transducer and wherein said compliant surface substantially surrounds said elongate transducer.
